(19) United States
(12) Patent Application Publication
(10) Pub. No.: US 2020/0392315 A1
(43) Pub. Date: Dec. 17, 2020

(54) RUBBER COMPOSITION FOR TIRE, AND PNEUMATIC TIRE

(71) Applicant: Toyo Tire Corporation, Itami-shi, Hyogo (JP)

(72) Inventor: Keisuke Iwakuni, Itami-shi (JP)

(73) Assignee: Toyo Tire Corporation, Itami-shi, Hyogo (JP)

(21) Appl. No.: 16/956,911

(22) PCT Filed: Jan. 28, 2019
(86) PCT No.: PCT/JP2019/002692

§ 371 Date: Jun. 22, 2020

(30) Foreign Application Priority Data
Jan. 31, 2018 (JP) 2018-014612

(51) Int. Cl. C08L 9/06; C07C 53/126; B60C 1/00; B60C 11/00

(52) U.S. Cl. CPC C08L 9/06; B60C 11/0008; B60C 1/0016; C07C 53/126

(57) ABSTRACT

A rubber composition for a tire comprising a rubber component containing natural rubber, styrene-butadiene rubber and butadiene rubber, silica, and an ether ester compound having HLB of 10 or less represented by the following formula (1):

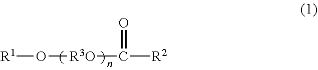

wherein R¹ and R² each independently represent a hydrocarbon group having from 1 to 30 carbon atoms, R³ represents an alkylene group having from 2 to 4 carbon atoms, n represents an average addition molar number, and 60 mass % or more of (R³O)ₙ comprises an oxyethylene group, is disclosed. Furthermore, a pneumatic tire having a tread comprising the rubber composition is disclosed.

### TECHNICAL FIELD

The present invention relates to a rubber composition for a tire, and a pneumatic tire using the same.

### BACKGROUND ART

For example, a rubber composition forming a tread of winter tires for Europe is required to improve wet performance as running performance on wet road surface together with snow performance as running performance on snowy road. Various investigations have been conventionally made, but it does not yet reach to sufficiently satisfy both performances

It is proposed in Patent Literature 1 that in order to improve on-ice performance together with wet performance in a rubber composition for studless tires, 10 parts by mass or more of silica are added and additionally glycerin monofatty acid ester and thermally expandable microcapsules are added, to 100 parts by mass of diene rubber. However, it is not described to add polyoxyalkylene alkyl ether fatty acid ester.

On the other hand, Patent Literature 2 discloses that in a rubber composition using a white filler, that is, silica, as a filler, polyoxyalkylene alkyl ether fatty acid ester is added. However, this literature adds the fatty acid ester in order to impart antistatic performance to a silica-added rubber composition, and does not describe that snow performance and wet performance can be improved by adding the fatty acid ester. Furthermore, the polyoxyalkylene alkyl ether fatty acid ester specifically used in Patent Literature 2 has high HLB and does not describe using polyoxyalkylene alkyl ether fatty acid ester having HLB of 10 or less.

### CITATION LIST

### Patent Literature

Patent Literature 1: JP-A-2016-023213

Patent Literature 2: JP-A-H10-330539

### SUMMARY OF INVENTION

### Technical Problem

An object of an embodiment of the present invention is to provide a rubber composition for a tire, that can improve snow performance and wet performance.

### Solution to Problem

The rubber composition for a tire according to an embodiment of the present invention comprises a rubber component containing natural rubber, styrene-butadiene rubber and butadiene rubber, silica and an ether ester compound having HLB of 10 or less represented by the following general formula (1):

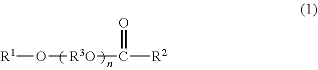

In the formula, R¹ and R² each independently represent a hydrocarbon group having from 1 to 30 carbon atoms, R³ represents an alkylene group having from 2 to 4 carbon atoms, n represents the average number of moles of oxyalkylene groups added, and 60 mass % or more of (R³O)ₙ comprises an oxyethylene group.

A pneumatic tire according to the embodiment of the present invention has a tread comprising the rubber composition.

### Advantageous Effects of Invention

According to the embodiment of the present invention, snow performance and wet performance can be improved by adding the ether ester compound.

### MODE FOR CARRYING OUT THE INVENTION

The rubber composition according to this embodiment comprises a rubber component comprising diene rubber, having added thereto silica and a specific ether ester compound.

The rubber component contains natural rubber (NR), styrene-butadiene rubber (SBR) and butadiene rubber (BR). The improvement effect in snow performance and wet performance can be enhanced by combining those. The natural rubber, styrene-butadiene rubber and butadiene rubber are not particularly limited, and various NR, SBR and BR generally used in a rubber composition for tires can be used. Those rubbers may be unmodified rubber and may be modified rubber. Furthermore, as the SBR, solution-polymerized SBR (SSBR) may be used and emulsion-polymerized SBR (ESBR) may be used.

SBR, BR and NR each having a functional group containing oxygen atom and/or nitrogen atom incorporated therein are exemplified as the modified rubbers, that is, modified SBR, modified BR and modified NR The modified rubbers have high polarity as compared with unmodified rubbers, and therefore can improve interaction with silica and the ether ester compound.

At least one selected from the group consisting of an amino group, an alkoxyl group, a hydroxyl group, an epoxy group, a carboxyl group and a carboxylic acid derivative group is exemplified as the functional group of the modified rubbers. The amino group may be not only a primary amino group, but may be a secondary or tertiary amino group. In the case of a secondary or tertiary amino group, the number of carbon atoms of the hydrocarbon group as a substituent is preferably 15 or less in total. As the alkoxyl group, a methoxy group, an ethoxy group, a propoxy group, a butoxy group and the like that are represented by —OA (wherein A is, for example, an alkyl group having from 1 to 4 carbon atoms) are exemplified. Furthermore, the alkoxyl group may be contained as an alkoxysilyl group (at least one of three hydrogens of a silyl group is substituted with an alkoxyl group) such as a trialkoxysilyl group, an alkyl dialkoxysilyl group or a dialkyl alkoxysilyl group. As the carboxylic acid derivative group, an ester group derived from carboxylic acid (carboxylic acid ester group) and an acid anhydride group comprising an anhydride of dicarboxylic acid such as maleic acid or phthalic acid are exemplified. As the carboxylic acid ester group, for example, an acrylate group (—O—CO—CH═CH₂) and/or a methacrylate group (—O—CO—C(CH₃)—CH₂) (hereinafter referred to as a (meth)acrylate group) are exemplified. As one embodiment, the functional group of the modified rubber may be at least one selected from the group consisting of an amino group, an alkoxyl group and a hydroxyl group. Those functional groups may be incorporated in at least one end of the diene rubber, or may be incorporated in a molecular chain. As the modified rubber, modified SBR and/or modified BR are preferably used, and modified SBR is more preferably used.

In the preferred one embodiment, 100 parts by mass of the rubber component contains from 10 to 60 parts by mass of natural rubber, from 20 to 70 parts by mass of styrene-butadiene rubber and from 10 to 50 parts by mass of butadiene rubber. More preferably, 100 parts by mass of the rubber component contains from 20 to 40 parts by mass of natural rubber, from 20 to 70 parts by mass of styrene-butadiene rubber and from 10 to 50 parts by mass of butadiene rubber, and may contain from 20 to 40 parts by mass of natural rubber, from 40 to 60 parts by mass of styrene-butadiene rubber and from 10 to 30 parts by mass of butadiene rubber.

The rubber component may be constituted of only natural rubber, styrene-butadiene rubber and butadiene rubber, but other rubbers, for example, nitrile rubber (NBR), chloroprene rubber (CR), butyl rubber (IIR), styrene-isoprene copolymer rubber, butadiene-isoprene copolymer rubber and styrene-isoprene-butadiene copolymer rubber, may be further added in a range that does not impair the original effect.

Silica as a filler is not particularly limited. For example, wet silica such as wet precipitated silica or wet gel process silica may be used. BET specific surface area of silica (measured according to BET method described in JIS K6430) is not particularly limited, and for example, may be from 100 to 300 m/g and may be from 150 to 250 m/g.

The amount of the silica added may be from 40 to 150 parts by mass and may be from 60 to 150 parts by mass, per 100 parts by mass of the rubber component. For enhancing the improvement effect of wet performance, the amount of the silica added is preferably from 70 to 150 parts by mass per 100 parts by mass of the rubber component. When the amount of the silica added is 150 parts by mass or less, deterioration of processability can be suppressed. In this embodiment, silica is used as a main filler. Preferably, more than 50 mass % of the filler is silica, and more preferably, more than 70 mass % of the filler is silica.

Silica may be used alone as the filler, but carbon black may be added together with silica. The carbon black is not particularly limited, and conventional various kinds can be used. For example, in the case of using in a tire tread rubber, SAF grade (N100 series), ISAF grade (N200 Series), HAF grade (N300 Series) and FEF grade (N500 Series) (those are ASTM grade) are preferably used. Carbon blacks of each grade can be used in one kind or as a mixture of two or more kinds. The amount of the carbon black added is not particularly limited. The amount may be 20 parts by mass or less and may be from 5 to 15 parts by mass, per 100 parts by mass of the rubber component.

An ether ester compound (preferably polyoxyalkylene alkyl ether fatty acid ester) having HLB of 10 or less represented by the following formula (1) is added to the rubber composition according to this embodiment. The ether ester compound shows plasticization effect in the rubber composition. It is therefore considered that viscosity during kneading the rubber composition is decreased and processability can be improved. By optimizing the proportion of an oxyalkylene group such that HLB of the ether ester compound is 10 or less, a coagulation temperature is decreased and the ether ester compound functions as a plasticizer in a polymer even at low temperature. As a result, it is considered that rubber flexibility at low temperature is maintained and snow performance is improved. Furthermore, it is considered that coagulation of the silica is relaxed by an oxyalkylene group as a hydrophilic group and wet performance by silica can be maximally expressed.

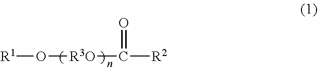

In the formula (1), R¹ and R² each independently represent a hydrocarbon group having from 1 to 30 carbon atoms. The number of carbon atoms of the hydrocarbon group is more preferably from 5 to 25 and still more preferably from 8 to 22, and may be from 10 to 20. The hydrocarbon group is preferably linear or branched saturated or unsaturated aliphatic hydrocarbon group. For example, an alkyl group or an alkenyl group is preferred. In one embodiment, R¹ is preferably an alkyl group or alkenyl group, having from 1 to 25 carbon atoms, and more preferably an alkyl group or alkenyl group, having from 8 to 20 carbon atoms. R² is preferably an alkyl group or alkenyl group, having from 8 to 25 carbon atoms, and more preferably an alkyl group or alkenyl group, having from 12 to 20 carbon atoms.

In the formula (1), R³ represents an alkylene group having from 2 to 4 carbon atoms, and n represents the average number of moles of oxyalkylene groups added. The alkylene group of R³ may be a straight chain and may be a branched chain. An oxyethylene group, an oxypropylene group, an oxybutylene group and the like are exemplified as the oxyalkylene group represented by R³O. (R³O)ₙ in the formula (1) is a polyoxyalkylene chain obtained by addition-polymerizing alkylene oxide having from 2 to 4 carbon atoms (for example, ethylene oxide, propylene oxide or butylene oxide). Polymerization form of alkylene oxide or the like is not particularly limited, and may be a homopolymer, a random copolymer or a block copolymer.

(R³O)ₙ in the formula (1) preferably comprises mainly an oxyethylene group, and 60 mass % or more of (R³O)ₙ preferably comprises an oxyethylene group. In other words, a polyoxyalkylene chain represented by (R³O)ₙ contains an oxyethylene group in an amount of preferably 60 mass % or more and more preferably 80 mass % or more (all oxyalkylene groups constituting the polyoxyalkylene chain is 100 mass %). Particularly preferably, the polyoxyalkylene chain contains 100 mass % of an oxyethylene group, that is, comprises only an oxyethylene group as shown in the following formula (2).

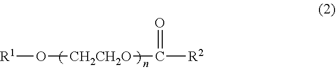

R¹, R² and n in the formula (2) are the same as R¹, R² and n in the formula (1).

The n showing the average number of moles of oxyalkylene groups added is a number that is set such that HLB of the ether ester compound is 10 or less. Although varying depending on the kind of R¹ and R², for example, n may be from 1 to 20, may be from 2 to 15 and may be from 3 to 10.

The HLB (hydrophilic-lipophilic balance) of the ether ester compound is 10 or less, more preferably from 3 to 10 and still more preferably from 4 to 8, in order to decrease the coagulation temperature at low temperature as described above. The HLB is a value calculated by the following Griffin's formula, and shows that the proportion of hydrophilic moiety occupied in the whole molecules is large and hydrophilicity is high, as the value is large.

HLB=−20×(molecular weight of hydrophilic moiety)/(whole molecular weight)

The molecular weight of the hydrophilic moiety in the formula is a molecular weight of a polyoxyalkylene chain represented by (R³O)ₙ.

The addition amount of the ether ester compound of the formula (1) is not particularly limited, but is preferably from 1 to 20 parts by mass and more preferably from 2 to 15 parts by mass, per 100 parts by mass of the rubber component. The amount may be from 3 to 10 parts by mass.

Other than the above components, various additives generally used in a rubber composition, such as a silane coupling agent, oil, zinc flower, stearic acid, an age resister, a wax, a vulcanizing agent and vulcanization accelerator, can be added to the rubber composition according to this embodiment.

The silane coupling agent includes sulfide silane and mercaptosilane. The amount of the silane coupling agent added is not particularly limited, but is preferably from 2 to 20 mass % based on the amount of the silica added.

Sulfur is preferably used as the vulcanizing agent. The amount of the vulcanizing agent added is not particularly limited, but is preferably from 0.1 to 10 parts by mass and more preferably from 0.5 to 5 parts by mass, per 100 parts by mass of the rubber component. The vulcanization accelerator includes various vulcanization accelerators such as sulfenamide type, thiuram type, thiazole type and guanidine type, and those can be used in one kind alone or as a mixture of two or more kinds. The amount of the vulcanization accelerator added is not particularly limited, but is preferably from 0.1 to 7 parts by mass and more preferably from 0.5 to 5 parts by mass, per 100 parts by mass of the rubber component.

The rubber composition according to this embodiment can be prepared by kneading the necessary components according to the conventional methods using a mixing machine generally used, such as Banbury mixer, a kneader or rolls. In other words, for example, additives other than a vulcanizing agent and a vulcanization accelerator are added to the rubber component together with silica and the ether ester compound, followed by mixing, in a first mixing step (non-productive mixing step). A vulcanizing agent and a vulcanization accelerator are then added to the mixture thus obtained, followed by mixing, in a final mixing step (productive mixing step). Thus, an unvulcanized rubber composition can be prepared.

The rubber composition according to this embodiment can be used in, for example, tires for various uses, such as for passenger cars or for heavy load of trucks or buses. The rubber composition is preferably used in a tread of a pneumatic tire, that is, is a rubber composition for a tire tread. In this embodiment, snow performance and wet performance are excellent as described above. Therefore, the rubber composition is suitably used as a rubber composition for a tread of winter tires (for example, winter tires for Europe).

The pneumatic tire according to one embodiment can be manufactured by preparing a tread rubber of a tire by an extruder for rubber using the rubber composition, combining the tread rubber with other tire members to prepare an unvulcanized tire (green tire) and then vulcanization molding the unvulcanized tire at a temperature of, for example, from 140 to 180° C. The tread rubber of a pneumatic tire includes a tread rubber comprising a two-layered structure of a cap rubber and a base rubber, and a single layer structure in which those are integrated. The tread rubber is preferably used as a rubber constituting a ground contact surface. That is, it is preferred that when the tread rubber has a single layer structure, the tread rubber comprises the rubber composition mentioned above, and when the tread rubber has a two-layered structure, the cap rubber comprises the rubber composition mentioned above.

### EXAMPLES

Examples of the present invention are described below, but the present invention is not construed as being limited to those examples.

[Synthesis of Ether Ester Compound]

Compounds 1 to 5 used in the examples and comparative examples were synthesized by the following methods.

[Compound 1]

0.1 g of a potassium hydroxide catalyst was added to 47 g (0.25 mol) of lauryl alcohol (manufactured by Tokyo Chemical Industry Co., Ltd.), 33 g (0.75 mol) of ethylene oxide (manufactured by Tokyo Chemical Industry Co., Ltd.) was injected in the resulting mixture while stirring at a temperature of from 110 to 120° C., and addition reaction was conducted. The reactant was transferred to a flask, and potassium hydroxide as a catalyst was neutralized with phosphoric acid. A phosphate was filtered out from the neutralized material, and 72 g (yield 90 mass %) of an adduct of lauryl alcohol with 3 mol of ethylene oxide was obtained. 60 g (0.19 mol) of the adduct of lauryl alcohol with 3 mol of ethylene oxide obtained, 56 g (0.2 mol) of oleic acid (manufactured by Tokyo Chemical Industry Co., Ltd.) and 0.7 g of dibutyltin oxide as a catalyst were weighed and dehydration esterification reaction was conducted at 225° C. while stirring under nitrogen injection. Thus, compound 1 was obtained. The compound 1 is an ether ester compound of the formula (2) wherein R¹=C₁₂H₂₅, R²=C₁₇H₃₃, n=3 and HLB=5.

[Compound 2]

0.1 g of a potassium hydroxide catalyst was added to 47 g (0.25 mol) of lauryl alcohol (manufactured by Tokyo Chemical Industry Co., Ltd.), 33 g (1.5 mol) of ethylene oxide (manufactured by Tokyo Chemical Industry Co., Ltd.) was injected in the resulting mixture while stirring at a temperature of from 110 to 120° C., and addition reaction was conducted. The reactant was transferred to a flask, and potassium hydroxide as a catalyst was neutralized with phosphoric acid. A phosphate was filtered out from the neutralized material, and 150 g (yield 84 mass %) of an adduct of lauryl alcohol with 6 mol of ethylene oxide was obtained. 135 g (0.19 mol) of the adduct of lauryl alcohol with 6 mol of ethylene oxide obtained, 56 g (0.2 mol) of oleic acid (manufactured by Tokyo Chemical Industry Co., Ltd.) and 0.7 g of dibutyltin oxide as a catalyst were weighed and dehydration esterification reaction was conducted at 225° C. while stirring under nitrogen injection. Thus, compound 2 was obtained. The compound 2 is an ether ester compound of the formula (2) wherein R=C₁₂H₂₅, R²=C₁₇H₃₃, n=6 and HLB=7.

[Compound 3]

0.1 g of a potassium hydroxide catalyst was added to 47 g (0.25 mol) of lauryl alcohol (manufactured by Tokyo Chemical Industry Co., Ltd.), 330 g (7.5 mol) of ethylene oxide (manufactured by Tokyo Chemical Industry Co., Ltd.) was injected in the resulting mixture while stirring at a temperature of from 110 to 120° C., and addition reaction was conducted. The reactant was transferred to a flask, and potassium hydroxide as a catalyst was neutralized with phosphoric acid. A phosphate was filtered out from the neutralized material, and 336 g (yield 76 mass %) of an adduct of lauryl alcohol with 30 mol of ethylene oxide was obtained. 200 g (0.11 mol) of the adduct of lauryl alcohol with 30 mol of ethylene oxide obtained, 34 g (0.12 mol) of oleic acid (manufactured by Tokyo Chemical Industry Co., Ltd.) and 0.7 g of dibutyltin oxide as a catalyst were weighed and dehydration esterification reaction was conducted at 225° C. while stirring under nitrogen injection. Thus, compound 3 was obtained. The compound 3 is an ether ester compound of the formula (2) wherein R¹=C₁₂H₂₅, R²—C₁₇H₃₃, n=30 and HLB=15.

[Compound 4]

0.1 g of a potassium hydroxide catalyst was added to 30 g (0.15 mol) of tridecyl alcohol (manufactured by Tokyo Chemical Industry Co., Ltd.), 46 g (1.05 mol) of ethylene oxide (manufactured by Tokyo Chemical Industry Co., Ltd.) was injected in the resulting mixture while stirring at a temperature of from 110 to 120° C., and addition reaction was conducted. The reactant was transferred to a flask, and potassium hydroxide as a catalyst was neutralized with phosphoric acid. A phosphate was filtered out from the neutralized material, and 64 g (yield 85 mass %) of an adduct of tridecyl alcohol with 7 mol of ethylene oxide was obtained. 60 g (0.12 mol) of the adduct of tridecyl alcohol with 7 mol of ethylene oxide obtained, 37 g (0.13 mol) of stearic acid (manufactured by Tokyo Chemical Industry Co., Ltd.) and 0.7 g of dibutyltin oxide as a catalyst were weighed and dehydration esterifcation reaction was conducted at 225° C. while stirring under nitrogen injection. Thus, compound 4 was obtained. The compound 4 is an ether ester compound of the formula (2) wherein R¹=C₁₃H₂₇, R²=CH₁₇H₃₅, n=7 and HLB=8.

[Compound 5]

0.1 g of a potassium hydroxide catalyst was added to 54 g (0.2 ml) of oleyl alcohol (manufactured by Tokyo Chemical Industry Co., Ltd.), 26 g (0.6 mol) of ethylene oxide (manufactured by Tokyo Chemical Industry Co., Ltd.) was injected in the resulting mixture while stirring at a temperature of from 110 to 120° C., and addition reaction was conducted. The reactant was transferred to a flask, and potassium hydroxide as a catalyst was neutralized with phosphoric acid. A phosphate was filtered out from the neutralized material, and 69 g (yield 90 mass %) of an adduct of oleyl alcohol with 3 mol of ethylene oxide was obtained. 58 g (0.15 mol) of the adduct of oleyl alcohol with 3 mol of ethylene oxide obtained, 47 g (0.165 mol) of stearic acid (manufactured by Tokyo Chemical Industry Co., Ltd.) and 0.7 g of dibutyltin oxide as a catalyst were weighed and dehydration esterification reaction was conducted at 225° C. while stirring under nitrogen injection. Thus, compound 5 was obtained. The compound 5 is an ether ester compound of the formula (2) wherein R¹=C₁₈H₃₅, R²=C₁₇H₃₅, n=3 and HLB=4.

[Preparation and Evaluation of Rubber Composition and Tire]

Banbury mixer was used. Compounding ingredients excluding sulfur and a vulcanization accelerator were added to a rubber component according to the formulations (parts by mass) shown in Table 1 below, followed by kneading, in a first mixing step (discharge temperature: 160° C.). Sulfur and a vulcanization accelerator were then added to the kneaded material obtained, followed by kneading, in a final mixing step (discharge temperature: 90° C.). Thus, rubber compositions were prepared. The details of each component in Table 1 are as follows.

NR: RSS #3

SSBR1: TUFDENE 1834 (solution-polymerized SBR) manufactured by Asahi Kasei Corporation

SSBR2: HPR340 (alkoxyl group and amino group-terminated modified solution-polymerized SBR) manufactured by JSR Corporation

BR: BR150B manufactured by Ube Industries, Ltd.

Carbon black: SFAST KH (N339) manufactured by Tokai Carbon Co., Ltd.

Silica: NIPSIL 1AQ manufactured by Tosoh Silica Corporation

Paraffin oil: JOMO PROCESS P200 manufactured by JX Nippon Oil &Sun-Energy Corporation

Aroma oil: JOMO PROCESS NC140 manufactured by JX Nippon Oil &Sun-Energy Corporation

Silane coupling agent: Si69 manufactured by Evonik Degussa

Stearic acid: LUNAC S-20 manufactured by Kao Corporation

Zinc flower: Zinc Flower #1 manufactured by Mitsui Mining & Smelting Co., Ltd.

Wax: OZOACE 0355 manufactured by Nippon Seiro Co., Ltd.

Age resister: NOCRAC 6C manufactured by Ouchi Shinko Chemical Industrial Co., Ltd.

Vulcanization accelerator: NOCCELER D manufactured by Ouchi Shinko Chemical Industrial Co., Ltd.

Sulfur: POWDERED SULFUR manufactured by Tsurumi Chemical Industry Co., Ltd.

Processability of each rubber composition obtained was evaluated. Furthermore, each rubber composition was used in a tread rubber, and a pneumatic radial tire (tire size: 215/45ZR17) was manufactured by vulcanization molding (160° C., 30 minutes) according to the conventional method. Snow performance and wet performance of the test tire obtained was evaluated. Each of measurement and evaluation methods is as follows.

Processability: Unvulcanized rubber was preheated at 100° C. for 1 minute and torque value after 4 minutes was measured in Mooney unit, using rotorless Mooney measuring instrument manufactured by Toyo Seiki Co., Ltd. according to JIS K6300. Inverse number of the measured value was indicated by an index as the value of Comparative Example 1 being 100. Mooney viscosity is low as the index is large, and large index means excellent processability.

Snow performance: Four test tires were mounted on a passenger car. ABS was operated from 60 km/hour running on snowy road and a braking distance when the speed was reduced to 20 kg/hour was measured (average value of n=10). Inverse number of the braking distance was indicated by an index as the value of Comparative Example 1 being 100. Braking distance is short as the index is large, and large index means that braking performance on snowy road surface is excellent.

Wet performance: Four test tires were mounted on a passenger car, and the passenger car was run on a road surface on which water was sprinkled in a depth of 2 to 3 mm, and wet grip performance was evaluated by measuring friction coefficient in 100 km/hour. The wet performance was indicated by an index as the value of friction coefficient of Comparative Example 1 being 100. Friction coefficient is high as the index is large, and large index means excellent wet grip performance.

TABLE 1

| | Com- | Com- | | | | | | | | | | |
|---|---|---|---|---|---|---|---|---|---|---|---|---|
| | para | para | | | | | | | | | | |
| | tive | tive | Exam- | Exam- | Exam- | Exam- | Exam- | Exam- | Exam- | Exam- | Exam- | Exam- |
| | Exam- | Exam- | ple | ple | ple | ple | ple | ple | ple | ple | ple | ple |
| | ple 1 | ple 2 | 1 | 2 | 3 | 4 | 5 | 6 | 7 | 8 | 9 | 10 |
| Formulation | | | | | | | | | | | | |
| (Parts by mass) | | | | | | | | | | | | |
| NR | 30 | 30 | 30 | 30 | 30 | 30 | 30 | 30 | 30 | 30 | 30 | 20 |
| SSBR1 | 50 | 50 | 50 | 50 | — | 50 | 50 | 50 | 50 | 50 | 20 | 70 |
| SSBR2 | — | — | — | — | 50 | — | — | — | — | — | — | — |
| BR | 20 | 20 | 20 | 20 | 20 | 20 | 20 | 20 | 20 | 20 | 50 | 10 |
| Carbon black | 10 | 10 | 10 | 10 | 10 | 10 | 10 | 10 | 10 | 10 | 10 | 10 |
| Silica | 90 | 90 | 50 | 90 | 90 | 140 | 90 | 140 | 90 | 90 | 90 | 90 |
| Paraffin oil | — | — | — | — | — | — | — | 30 | — | — | — | — |
| Aroma oil | 30 | 30 | 15 | 30 | 30 | 60 | 30 | 30 | 30 | 30 | 30 | 30 |
| Silane coupling agent | 9 | 9 | 5 | 9 | 9 | 14 | 9 | 14 | 9 | 9 | 9 | 9 |
| Stearic acid | 2 | 2 | 2 | 2 | 2 | 2 | 2 | 2 | 2 | 2 | 2 | |
| Zinc flower | 2 | 2 | 2 | 2 | 2 | 2 | 2 | 2 | 2 | 2 | 2 | 2 |
| Wax | 2 | 2 | 2 | 2 | 2 | 2 | 2 | 2 | 2 | 2 | 2 | 2 |
| Age resister | 2 | 2 | 2 | 2 | 2 | 2 | 2 | 2 | 2 | 2 | 2 | 2 |
| Compound 1 (HLB = 4) | — | — | 5 | 5 | 5 | 10 | — | 5 | — | — | 5 | 5 |
| Compound 2 (HLB = 7) | — | — | — | — | — | — | 5 | — | — | — | — | — |
| Compound 3 (HLB = 15) | — | 5 | — | — | — | — | — | — | — | — | — | — |
| Compound 4 (HLB = 8) | — | — | — | — | — | — | — | — | 5 | — | — | — |
| Compound 5 (HLB = 5) | — | — | — | — | — | — | — | — | — | 5 | — | — |
| Vulcanization accelerator | 15 | 1.5 | 1.5 | 1.5 | 1.5 | 1.5 | 1.5 | 1.5 | 1.5 | 1.5 | 1.5 | 1.5 |
| Sulfur | 2 | 2 | 2 | 2 | 2 | 2 | 2 | 2 | 2 | 2 | 2 | 2 |
| Evaluation (Index) | | | | | | | | | | | | |
| Processability | 100 | 100 | 105 | 100 | 95 | 90 | 95 | 100 | 102 | 103 | 102 | 105 |
| Snow performance | 100 | 80 | 105 | 110 | 115 | 110 | 108 | 115 | 105 | 106 | 115 | 110 |
| Wet performance | 100 | 100 | 103 | 105 | 108 | 115 | 108 | 105 | 105 | 104 | 105 | 110 |

The results are shown in Table 1. As compared with Comparative Example 1, improvement effect in snow performance was not obtained in Comparative Example 2 in which an ether ester compound having high HLB (compound 3) was added. On the other hand, in Examples 1 to 10 in which ether ester compounds having 10 or less of HLB (compounds 1, 2, 4 and 5) were added, both snow performance and wet performance were improved as compared with Comparative Example 1. Furthermore, from the comparison between Example 1 and Examples 2 and 4, snow performance and wet performance were further improved by increasing the amount of silica added.

Some embodiments of the present invention are described above, but those embodiments are merely shown as examples and are not intended to limit the scope of the invention. Those embodiments can be carried out in other various forms, and various omissions, rewriting and changes can be made in a range that does not deviate the gist of the invention. Those embodiments and their omissions, rewriting and changes are included in the scope and gist of the invention, and additionally included in the inventions recited in the scope of claims and their equivalent scopes.

1. A rubber composition for a tire comprising
a rubber component containing natural rubber, styrene-butadiene rubber and butadiene rubber,
silica, and
an ether ester compound having HLB of 10 or less represented by the following general formula (1): 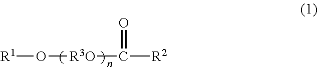
wherein R¹ and R² each independently represent a hydrocarbon group having from 1 to 30 carbon atoms, R³ represents an alkylene group having from 2 to 4 carbon atoms, n represents an average number of moles of oxyalkylene groups added, and 60 mass % or more of (R³O)ₙ comprises an oxyethylene group.

2. The rubber composition for a tire according to claim 1, wherein the amount of the silica added is from 70 to 150 parts by mass per 100 parts by mass of the rubber component.

3. The rubber composition for a tire according to claim 1, wherein the amount of the ether ester compound added is from 1 to 20 parts by mass per 100 parts by mass of the rubber component.

4. The rubber composition for a tire according to claim 1, wherein the styrene-butadiene rubber contains modified styrene-butadiene rubber having incorporated therein a functional group containing oxygen atom and/or nitrogen atom.

5. The rubber composition for a tire according to claim 1, wherein 100 parts by mass of the rubber component contains from 10 to 60 parts by mass of the natural rubber, from 20 to 70 parts by mass of the styrene-butadiene rubber and from 10 to 50 parts by mass of the butadiene rubber.

6. A pneumatic tire having a tread comprising the rubber composition according to claim 1.

7. The pneumatic tire according to claim 6, which is a winter tire.
